(19) United States
(12) Patent Application Publication
(10) Pub. No.: US 2005/0100933 A1
(43) Pub. Date: May 12, 2005

(54) Breast cancer survival and recurrence

(75) Inventors: Mark G. Erlander, Encinitas, CA (US); Xiao-Jun Ma, San Diego, CA (US); Wei Wang, San Marcos, CA (US); James L. Wittliff, Louisville, KY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arcturus Bioscience, Inc., Mountain View, CA (US)

(21) Appl. No.: 10/871,869

(22) Filed: Jun. 18, 2004

Related U.S. Application Data
(60) Provisional application No. 60/545,810, filed on Feb. 18, 2004.
(60) Provisional application No. 60/479,963, filed on Jun. 18, 2003.

(51) Int. Cl. C12Q 1/68; C12P 19/34

(52) U.S. Cl. 435/6; 435/91.2

(57) ABSTRACT

The invention provides for the identification and use of gene expression profiles, or patterns, with clinical relevance to breast cancer. In particular, the invention provides the identities of genes that are correlated with patient survival and breast cancer recurrence. The gene expression profiles may be embodied in nucleic acid expression, protein expression, or other expression formats and used to predict the survival of subjects afflicted with breast cancer and to predict breast cancer recurrence and. The profiles may also be used in the study and/or diagnosis of breast cancer cells and tissue, including the grading of invasive breast cancer, as well as for the study and/or determination of prognosis of a patient. When used for diagnosis or prognosis, the profiles may be used to determine the treatment of breast cancer based upon the likelihood of life expectancy and recurrence.

### RELATED APPLICATIONS

This application claims benefit of priority from U.S. Provisional Patent Application 60/479,963, filed Jun. 18, 2003, and 60/545,810, filed Feb. 18, 2004, both of which are incorporated by reference as if fully set forth.

### FIELD OF THE INVENTION

The invention relates to the identification and use of gene expression profiles, or patterns, with clinical relevance to breast cancer. In particular, the invention provides the identities of genes that are correlated with patient survival and breast cancer recurrence. The gene expression profiles, whether embodied in nucleic acid expression, protein expression, or other expression formats, may be used to predict the survival of subjects afflicted with breast cancer and to predict breast cancer recurrence and. The profiles may also be used in the study and/or diagnosis of breast cancer cells and tissue, including the grading of invasive breast cancer, as well as for the study and/or determination of prognosis of a patient. When used for diagnosis or prognosis, the profiles are used to determine the treatment of breast cancer based upon the likelihood of life expectancy and recurrence.

### BACKGROUND OF THE INVENTION

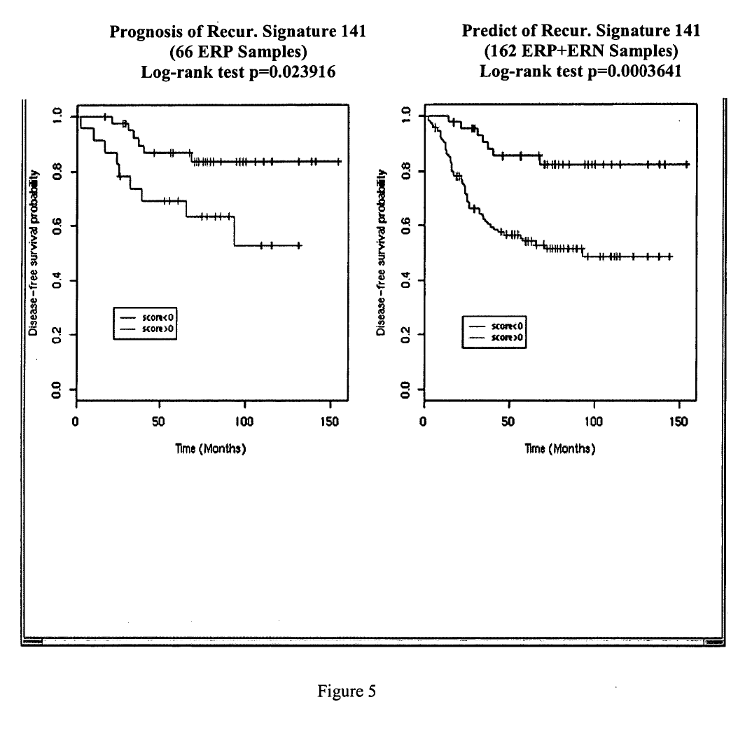

Breast cancer is by far the most common cancer among women. Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. An estimated 2,167,000 women in the United States are currently living with the disease (National Cancer Institute, Surveillance Epidemiology and End Results (NCI SEER) program, Cancer Statistics Review (CSR), www-seer.ims.nci.nih.gov/Publications/CSR1973 (1998)). Based on cancer rates from 1995 through 1997, a report from the National Cancer Institute (NCI) estimates that about 1 in 8 women in the United States (approximately 12.8 percent) will develop breast cancer during her lifetime (NCI's Surveillance, Epidemiology, and End Results Program (SEER) publication SEER Cancer Statistics Review 1973-1997). Breast cancer is the second most common form of cancer, after skin cancer, among women in the United States. An estimated 250,100 new cases of breast cancer are expected to be diagnosed in the United States in 2001. Of these, 192,200 new cases of more advanced (invasive) breast cancer are expected to occur among women (an increase of 5% over last year), 46,400 new cases of early stage (in situ) breast cancer are expected to occur among women (up 9% from last year), and about 1,500 new cases of breast cancer are expected to be diagnosed in men (Cancer Facts & Figures 2001 American Cancer Society). An estimated 40,600 deaths (40,300 women, 400 men) from breast cancer are expected in 2001. Breast cancer ranks second only to lung cancer among causes of cancer deaths in women. Nearly 86% of women who are diagnosed with breast cancer are likely to still be alive five years later, though 24% of them will die of breast cancer after 10 years, and nearly half (47%) will die of breast cancer after 20 years.

Every woman is at risk for breast cancer. Over 70 percent of breast cancers occur in women who have no identifiable risk factors other than age (U.S. General Accounting Office. Breast Cancer, 1971-1991: Prevention, Treatment and Research. GAO/PEMD-92-12; 1991). Only 5 to 10% of breast cancers are linked to a family history of breast cancer (Henderson I C, Breast Cancer. In: Murphy G P, Lawrence W L, Lenhard R E (eds). Clinical Oncology. Atlanta, Ga.: American Cancer Society; 1995:198-219).

Each breast has 15 to 20 sections called lobes. Within each lobe are many smaller lobules. Lobules end in dozens of tiny bulbs that can produce milk. The lobes, lobules, and bulbs are all linked by thin tubes called ducts. These ducts lead to the nipple in the center of a dark area of skin called the areola. Fat surrounds the lobules and ducts. There are no muscles in the breast, but muscles lie under each breast and cover the ribs. Each breast also contains blood vessels and lymph vessels. The lymph vessels carry colorless fluid called lymph, and lead to the lymph nodes. Clusters of lymph nodes are found near the breast in the axilla (under the arm), above the collarbone, and in the chest.

Breast tumors can be either benign or malignant. Benign tumors are not cancerous, they do not spread to other parts of the body, and are not a threat to life. They can usually be removed, and in most cases, do not come back. Malignant tumors are cancerous, and can invade and damage nearby tissues and organs. Malignant tumor cells may metastasize, entering the bloodstream or lymphatic system. When breast cancer cells metastasize outside the breast, they are often found in the lymph nodes under the arm (axillary lymph nodes). If the cancer has reached these nodes, it means that cancer cells may have spread to other lymph nodes or other organs, such as bones, liver, or lungs.

Major and intensive research has been focused on early detection, treatment and prevention. This has included an emphasis on determining the presence of precancerous or cancerous ductal epithelial cells. These cells are analyzed, for example, for cell morphology, for protein markers, for nucleic acid markers, for chromosomal abnormalities, for biochemical markers, and for other characteristic changes that would signal the presence of cancerous or precancerous cells. This has led to various molecular alterations that have been reported in breast cancer, few of which have been well characterized in human clinical breast specimens. Molecular alterations include presence/absence of estrogen and progesterone steroid receptors, HER-2 expression/amplification (Mark H F, et al. HER-2/neu gene amplification in stages I-IV breast cancer detected by fluorescent in situ hybridization. Genet Med; 1(3):98-103 1999), Ki-67 (an antigen that is present in all stages of the cell cycle except GO and used as a marker for tumor cell proliferation, and prognostic markers (including oncogenes, tumor suppressor genes, and angiogenesis markers) like p53, p27, Cathepsin D, pS2, multi-drug resistance (MDR) gene, and CD31.

van't Veer et al. (Nature 415:530-536, 2002) describe gene expression profiling of clinical outcome in breast cancer. They identified genes expressed in breast cancer tumors, the expression levels of which correlated either with patients afflicted with distant metastases within 5 years or with patients that remained metastasis-free after at least 5 years.

Ramaswamy et al. (Nature Genetics 33:49-54, 2003) describe the identification of a molecular signature of metastasis in primary solid tumors. The genes of the signature were identified based on gene expression profiles of 12 metastatic adenocarcinoma nodules of diverse origin (lung, breast, prostate, colorectal, uterus) compared to expression profiles of 64 primary adenocarcinomas representing the same spectrum of tumor types from different individuals. A 128 gene set was identified.

Both of the above described approaches, however, utilize heterogeneous populations of cells found in a tumor sample to obtain information on gene expression patterns. The use of such populations may result in the inclusion or exclusion of multiple genes that are differentially expressed in cancer cells. The gene expression patterns observed by the above described approaches may thus provide little confidence that the differences in gene expression are meaningfully associated with breast cancer recurrence or survival.

Citation of documents herein is not intended as an admission that any is pertinent prior art. All statements as to the date or representation as to the contents of documents is based on the information available to the applicant and does not constitute any admission as to the correctness of the dates or contents of the documents.

### BRIEF SUMMARY OF THE INVENTION

The present invention relates to the identification and use of gene expression patterns (or profiles or “signatures”) which are clinically relevant to breast cancer. In particular, the identities of genes that are correlated with patient survival and breast cancer recurrence are provided. The gene expression profiles, whether embodied in nucleic acid expression, protein expression, or other expression formats, may be used to predict survival of subjects afflicted with breast cancer and the likelihood of breast cancer recurrence.

The invention thus provides for the identification and use of gene expression patterns (or profiles or “signatures”) which correlate with (and thus able to discriminate between) patients with good or poor survival outcomes. In one embodiment, the invention provides patterns that are able to distinguish patients with estrogen receptor (ER) positive breast tumors into those with a survival outcome poorer than that of patients with ER negative breast tumors and those with a better survival outcome than that of patients with ER positive breast tumors. These patterns are thus able to distinguish patients with ER positive breast tumors into at least two subtypes.

The invention also provides for the identification and use of gene expression patterns which correlate with the recurrence of breast cancer at the same location and/or in the form of metastases. The pattern is able to distinguish patients with breast cancer into at least those with good or poor survival outcomes.

In another aspect of the invention, the ability to identify the grade of invasive breast cancer by gene expression patterns of the invention is provided. In particular, gene expression patterns in a cell containing sample that distinguish “high-grade” (or “grade 3”) invasive breast tumors from “low-grade” (or grades “1” and “2”) invasive breast tumors are provided. The invention thus permits the distinguishing (or grading) of a subject's invasive tumors into two types which may be differentially treated based on the expected outcome associated with each type.

The present invention provides a non-subjective means for the identification of patients with breast cancer as likely to have a good or poor survival outcome by assaying for the expression patterns disclosed herein. Thus where subjective interpretation may have been previously used to determine the prognosis and/or treatment of breast cancer patients, the present invention provides objective gene expression patterns, which may used alone or in combination with subjective criteria to provide a more accurate assessment of breast cancer patient outcomes, including survival and the recurrence of cancer. The expression patterns of the invention thus provide a means to determine breast cancer prognosis. Furthermore, the expression patterns can also be used as a means to assay small, node negative tumors that are not readily assayed by other means.

The gene expression patterns comprise one or more than one gene capable of discriminating between breast cancer outcomes with significant accuracy. The gene(s) are identified as correlated with various breast cancer outcomes such that the levels of their expression are relevant to a determination of the preferred treatment protocols, of a breast cancer patient. Thus in one aspect, the invention provides a method to determine the outcome of a subject afflicted with, or suspected of having, breast cancer by assaying a cell containing sample from said subject for expression of one or more than one gene disclosed herein as correlated with breast cancer outcomes.

Gene expression patterns of the invention are identified as described below. Generally, a large sampling of the gene expression profile of a sample is obtained through quantifying the expression levels of mRNA corresponding to many genes. This profile is then analyzed to identify genes, the expression of which are positively, or negatively, correlated, with a breast cancer outcome. An expression profile of a subset of human genes may then be identified by the methods of the present invention as correlated with a particular breast cancer outcome. The use of multiple samples increases the confidence which a gene may be believed to be correlated with a particular survival outcome. Without sufficient confidence, it remains unpredictable whether a particular gene is actually correlated with a breast cancer outcome and also unpredictable whether a particular gene may be successfully used to identify the outcome for a breast cancer patient.

A profile of genes that are highly correlated with one outcome relative to another may be used to assay an sample from a subject afflicted with, or suspected of having, breast cancer to predict the outcome of the subject from whom the sample was obtained. Such an assay may be used as part of a method to determine the therapeutic treatment for said subject based upon the breast cancer outcome identified.

The correlated genes may be used singly with significant accuracy or in combination to increase the ability to accurately correlating a molecular expression phenotype with a breast cancer outcome. This correlation is a way to molecularly provide for the determination of survival outcomes as disclosed herein. Additional uses of the correlated gene(s) are in the classification of cells and tissues; determination of diagnosis and/or prognosis; and determination and/or alteration of therapy.

The ability to discriminate is conferred by the identification of expression of the individual genes as relevant and not by the form of the assay used to determine the actual level of expression. An assay may utilize any identifying feature of an identified individual gene as disclosed herein as long as the assay reflects, quantitatively or qualitatively, expression of the gene in the “transcriptome” (the transcribed fraction of genes in a genome) or the “proteome” (the translated fraction of expressed genes in a genome). Identifying features include, but are not limited to, unique nucleic acid sequences used to encode (DNA), or express (RNA), said gene or epitopes specific to, or activities of, a protein encoded by said gene. All that is required is the identity of the gene(s) necessary to discriminate between breast cancer outcomes and an appropriate cell containing sample for use in an expression assay.

In one embodiment, the invention provides for the identification of the gene expression patterns by analyzing global, or near global, gene expression from single cells or homogenous cell populations which have been dissected away from, or otherwise isolated or purified from, contaminating cells beyond that possible by a simple biopsy. Because the expression of numerous genes fluctuate between cells from different patients as well as between cells from the same patient sample, multiple data from expression of individual genes and gene expression patterns are used as reference data to generate models which in turn permit the identification of individual gene(s), the expression of which are most highly correlated with particular breast cancer outcomes.

In another aspect, the invention provides physical and methodological means for detecting the expression of gene(s) identified by the models generated by individual expression patterns. These means may be directed to assaying one or more aspect of the DNA template(s) underlying the expression of the gene(s), of the RNA used as an intermediate to express the gene(s), or of the proteinaceous product expressed by the gene(s).

In a further aspect, the gene(s) identified by a model as capable of discriminating between breast cancer outcomes may be used to identify the cellular state of an unknown sample of cell(s) from the breast. Preferably, the sample is isolated via non-invasive means. The expression of said gene(s) in said unknown sample may be determined and compared to the expression of said gene(s) in reference data of gene expression patterns correlated with breast cancer outcomes. Optionally, the comparison to reference samples may be by comparison to the model(s) constructed based on the reference samples.

One advantage provided by the present invention is that contaminating, non-breast cells (such as infiltrating lymphocytes or other immune system cells) are not present to possibly affect the genes identified or the subsequent analysis of gene expression to identify the survival outcomes of patients with breast cancer. Such contamination is present where a biopsy is used to generate gene expression profiles.

In another aspect, the invention provides the identification and use of four gene sequences the expression of which are significantly associated with tumor recurrence. Elevated expression of each one of the four gene sequences is correlated with increased likelihood of tumor recurrence and decreased patient survival. Therefore, the expression of each of these gene sequences may be used in the same manner as described herein for gene expression patterns.

The first set of sequences is that of mitotic spindle associated protein (also known as mitotic spindle coiled-coil related protein, ASTRIN or DEEPEST). Human DEEPEST protein has been characterized by Mack et al. (Proc Natl Acad Sci USA. 2001 98(25): 14434-9).

The second set of sequences is that of the “Rac GTPase activating protein 1” (RACGAP1).

The third set of sequences is that of the “zinc finger protein 145” or “PLZF” (Kruppel-like zinc finger protein, expressed in promyelocytic leukemia) which is also referred to as ZNF145.

The fourth set of sequences is that of “MS4A7” (membrane-spanning 4-domains, subfamily A, member 7).

While the present invention is described mainly in the context of human breast cancer, it may be practiced in the context of breast cancer of any animal known to be potentially afflicted by breast cancer. Preferred animals for the application of the present invention are mammals, particularly those important to agricultural applications (such as, but not limited to, cattle, sheep, horses, and other “farm animals”), animal models of breast cancer, and animals for human companionship (such as, but not limited to, dogs and cats).

### DETAILED DESCRIPTION OF THE INVENTION

Definitions of terms as used herein:

A gene expression “pattern” or “profile” or “signature” refers to the relative expression of a gene between two or more breast cancer survival outcomes which is correlated with being able to distinguish between said outcomes.

A “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

The terms “correlate” or “correlation” or equivalents thereof refer to an association between expression of one or more genes and a physiologic state of a breast cell to the exclusion of one or more other state as identified by use of the methods as described herein. A gene may be expressed at higher or lower levels and still be correlated with one or more breast cancer state or outcome.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence.

By corresponding is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17). Methods for amplifying mRNA are generally known in the art, and include reverse transcription PCR (RT-PCR) and those described in U.S. patent application Ser. No. 10/062,857 (filed on Oct. 25, 2001), as well as U.S. Provisional Patent Application 60/298,847 (filed Jun. 15, 2001) and 60/257,801 (filed Dec. 22, 2000), all of which are hereby incorporated by reference in their entireties as if fully set forth. Another method which may be used is quantitative PCR (or Q-PCR). Alternatively, RNA may be directly labeled as the corresponding cDNA by methods known in the art.

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm2, more preferably at least about 100/cm2, even more preferably at least about 500/cm2, but preferably below about 1,000/cm2. Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of a sample polynucleotides can be determined based on their binding to a particular position in the microarray.

Because the invention relies upon the identification of genes that are over- or under-expressed, one embodiment of the invention involves determining expression by hybridization of mRNA, or an amplified or cloned version thereof, of a sample cell to a polynucleotide that is unique to a particular gene sequence. Preferred polynucleotides of this type contain at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Even more preferred are polynucleotides of at least or about 50, at least or about 100, at least about or 150, at least or about 200, at least or about 250, at least or about 300, at least or about 350, or at least or about 400 basepairs of a gene sequence that is not found in other gene sequences. The term “about” as used in the preceding sentence refers to an increase or decrease of 10% from the stated numerical value. Such polynucleotides may also be referred to as polynucleotide probes that are capable of hybridizing to sequences of the genes, or unique portions thereof, described herein. Preferably, the sequences are those of mRNA encoded by the genes, the corresponding cDNA to such mRNAs, and/or amplified versions of such sequences. In preferred embodiments of the invention, the polynucleotide probes are immobilized on an array, other devices, or in individual spots that localize the probes.

Alternatively, and in another embodiment of the invention, gene expression may be determined by analysis of expressed protein in a cell sample of interest by use of one or more antibodies specific for one or more epitopes of individual gene products (proteins) in said cell sample. Such antibodies are preferably labeled to permit their easy detection after binding to the gene product.

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

As used herein, a “breast tissue sample” or “breast cell sample” refers to a sample of breast tissue or fluid isolated from an individual suspected of being afflicted with, or at risk of developing, breast cancer. Such samples are primary isolates (in contrast to cultured cells) and may be collected by any non-invasive means, including, but not limited to, ductal lavage, fine needle aspiration, needle biopsy, the devices and methods described in U.S. Pat. No. 6,328,709, or any other suitable means recognized in the art. Alternatively, the “sample” may be collected by an invasive method, including, but not limited to, surgical biopsy.

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of gene expression, mutations in non-coding regions of genes as disclosed herein may also be assayed in the practice of the invention.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

Unless defined otherwise all technical and scientific terms used herein have the same meaning as commonly understood to one of ordinary skill in the art to which this invention belongs.

### Specific Embodiments

The present invention relates to the identification and use of gene expression patterns (or profiles or “signatures”) which discriminate between (or are correlated with) breast cancer survival and recurrence outcomes in a subject. Such patterns may be determined by the methods of the invention by use of a number of reference cell or tissue samples, such as those reviewed by a pathologist of ordinary skill in the pathology of breast cancer, which reflect breast cancer cells as opposed to normal or other non-cancerous cells. The outcomes experienced by the subjects from whom the samples may be correlated with expression data to identify patterns that correlate with the outcomes. Because the overall gene expression profile differs from person to person, cancer to cancer, and cancer cell to cancer cell, correlations between certain cells and genes expressed or underexpressed may be made as disclosed herein to identify genes that are capable of discriminating between breast cancer outcomes.

The present invention may be practiced with any number of the genes believed, or likely to be, differentially expressed with respect to breast cancer outcomes. The identification may be made by using expression profiles of various homogenous breast cancer cell populations, which were isolated by microdissection, such as, but not limited to, laser capture microdissection (LCM) of 100-1000 cells. The expression level of each gene of the expression profile may be correlated with a particular outcome. Alternatively, the expression levels of multiple genes may be clustered to identify correlations with particular outcomes.

Genes with significant correlations to breast cancer survival or recurrence outcomes may be used to generate models of gene expressions that would maximally discriminate between outcomes. Alternatively, genes with significant correlations may be used in combination with genes with lower correlations without significant loss of ability to discriminate between outcomes. Such models may be generated by any appropriate means recognized in the art, including, but not limited to, cluster analysis, supported vector machines, neural networks or other algorithm known in the art. The models are capable of predicting the classification of a unknown sample based upon the expression of the genes used for discrimination in the models. “Leave one out” cross-validation may be used to test the performance of various models and to help identify weights (genes) that are uninformative or detrimental to the predictive ability of the models. Cross-validation may also be used to identify genes that enhance the predictive ability of the models.

The gene(s) identified as correlated with particular breast cancer outcomes by the above models provide the ability to focus gene expression analysis to only those genes that contribute to the ability to identify a subject as likely to have a particular outcome relative to another. The expression of other genes in a breast cancer cell would be relatively unable to provide information concerning, and thus assist in the discrimination of, a breast cancer outcome.

As will be appreciated by those skilled in the art, the models are highly useful with even a small set of reference gene expression data and can become increasingly accurate with the inclusion of more reference data although the incremental increase in accuracy will likely diminish with each additional datum. The preparation of additional reference gene expression data using genes identified and disclosed herein for discriminating between different outcomes in breast cancer is routine and may be readily performed by the skilled artisan to permit the generation of models as described above to predict the status of an unknown sample based upon the expression levels of those genes.

To determine the (increased or decreased) expression levels of genes in the practice of the present invention, any method known in the art may be utilized. In one preferred embodiment of the invention, expression based on detection of RNA which hybridizes to the genes identified and disclosed herein is used. This is readily performed by any RNA detection or amplification+detection method known or recognized as equivalent in the art such as, but not limited to, reverse transcription-PCR, the methods disclosed in U.S. patent application Ser. No. 10/062,857 (filed on Oct. 25, 2001) as well as U.S. Provisional Patent Application 60/298,847 (filed Jun. 15, 2001) and 60/257,801 (filed Dec. 22, 2000), and methods to detect the presence, or absence, of RNA stabilizing or destabilizing sequences.

Alternatively, expression based on detection of DNA status may be used. Detection of the DNA of an identified gene as methylated or deleted may be used for genes that have decreased expression in correlation with a particular breast cancer outcome. This may be readily performed by PCR based methods known in the art, including, but not limited to, Q-PCR. Conversely, detection of the DNA of an identified gene as amplified may be used for genes that have increased expression in correlation with a particular breast cancer outcome. This may be readily performed by PCR based, fluorescent in situ hybridization (FISH) and chromosome in situ hybridization (CISH) methods known in the art.

Expression based on detection of a presence, increase, or decrease in protein levels or activity may also be used. Detection may be performed by any immunohistochemistry (IHC) based, blood based (especially for secreted proteins), antibody (including autoantibodies against the protein) based, exfoliate cell (from the cancer) based, mass spectroscopy based, and image (including used of labeled ligand) based method known in the art and recognized as appropriate for the detection of the protein. Antibody and image based methods are additionally useful for the localization of tumors after determination of cancer by use of cells obtained by a non-invasive procedure (such as ductal lavage or fine needle aspiration), where the source of the cancerous cells is not known. A labeled antibody or ligand may be used to localize the carcinoma(s) within a patient.

A preferred embodiment using a nucleic acid based assay to determine expression is by immobilization of one or more sequences of the genes identified herein on a solid support, including, but not limited to, a solid substrate as an array or to beads or bead based technology as known in the art. Alternatively, solution based expression assays known in the art may also be used. The immobilized gene(s) may be in the form of polynucleotides that are unique or otherwise specific to the gene(s) such that the polynucleotide would be capable of hybridizing to a DNA or RNA corresponding to the gene(s). These polynucleotides may be the full length of the gene(s) or be short sequences of the genes (up to one nucleotide shorter than the full length sequence known in the art by deletion from the 5′ or 3′ end of the sequence) that are optionally minimally interrupted (such as by mismatches or inserted non-complementary basepairs) such that hybridization with a DNA or RNA corresponding to the gene(s) is not affected. Preferably, the polynucleotides used are from the 3′ end of the gene. Polynucleotides containing mutations relative to the sequences of the disclosed genes may also be used so long as the presence of the mutations still allows hybridization to produce a detectable signal.

The immobilized gene(s) may be used to determine the state of nucleic acid samples prepared from sample breast cell(s) for which the outcome of the sample's subject (e.g. patient from whom the sample is obtained) is not known or for confirmation of an outcome that is already assigned to the sample's subject. Without limiting the invention, such a cell may be from a patient with breast cancer or alternatively suspected of being afflicted with, or at risk of developing, breast cancer. The immobilized polynucleotide(s) need only be sufficient to specifically hybridize to the corresponding nucleic acid molecules derived from the sample under suitable conditions. While even a single correlated gene sequence may to able to provide adequate accuracy in discriminating between two breast cancer outcomes, two or more, three or more, four or more, five or more, six or more, seven or more, eight or more, nine or more, ten or more, or eleven or more of the genes identified herein may be used as a subset capable of discriminating may be used in combination to increase the accuracy of the method. The invention specifically contemplates the selection of more than one, two or more, three or more, four or more, five or more, six or more, seven or more, eight or more, nine or more, ten or more, or eleven or more of the genes disclosed in the tables and figures herein for use as a subset in the identification of breast cancer survival outcome.

Of course 15 or more, 20 or more, 30 or more, 40 or more, 50 or more, 60 or more, 70 or more, 80 or more, 90 or more, 100 or more, 110 or more, 120 or more, 130 or more, 140 or more, or all the genes provided in Tables 2, 3, and/or 4 below may be used. “CloneID” as used in the context of Tables 2, 3, and 4 as well as the present invention refers to the IMAGE Consortium clone ID number of each gene, the sequences of which are hereby incorporated by reference in their entireties as they are available from the Consortium at http://image.llnl.gov/ as accessed on the filing date of the present application. Also provided in the tables are GenBank accession numbers which are comprised of letters, numbers and optionally underscores. P value refers to values assigned as described in the Examples below. The indications of “E-xx” where “xx” is a two digit number refers to alternative notation for exponential figures where “E-xx” is “10-xx”. Thus in combination with the numbers to the left of “E-xx”, the value being represented is the numbers to the left times 10-xx. Description provides a brief identifier of what the gene encodes.

Genes with a correlation identified by a p value below or about 0.02, below or about 0.01, below or about 0.005, or below or about 0.001 are preferred for use in the practice of the invention. The present invention includes the use of genes that identify different ER positive subtypes and breast cancer recurrence and invasive tumor grade to permit simultaneous identification of breast cancer survival outcome of a patient based upon assaying a breast cancer sample from said patient.

In embodiments where only one or a few genes are to be analyzed, the nucleic acid derived from the sample breast cancer cell(s) may be preferentially amplified by use of appropriate primers such that only the genes to be analyzed are amplified to reduce contaminating background signals from other genes expressed in the breast cell. Alternatively, and where multiple genes are to be analyzed or where very few cells (or one cell) is used, the nucleic acid from the sample may be globally amplified before hybridization to the immobilized polynucleotides. Of course RNA, or the cDNA counterpart thereof may be directly labeled and used, without amplification, by methods known in the art.

The above assay embodiments may be used in a number of different ways to identify or detect the invasive breast cancer grade, if any, of a breast cancer cell sample from a patient. In many cases, this would reflect a secondary screen for the patient, who may have already undergone mammography or physical exam as a primary screen. If positive, the subsequent needle biopsy, ductal lavage, fine needle aspiration, or other analogous methods may provide the sample for use in the above assay embodiments. The present invention may be used in combination with non-invasive protocols, such as ductal lavage or fine needle aspiration, to prepare a breast cell sample.

The present invention provides a more objective set of criteria, in the form of gene expression profiles of a discrete set of genes, to discriminate (or delineate) between breast cancer outcomes. In particularly preferred embodiments of the invention, the assays are used to discriminate between good and poor outcomes within 5, or about 5, years after surgical intervention to remove breast cancer tumors or within about 95 months after surgical intervention to remove breast cancer tumors. Comparisons that discriminate between outcomes after about 10, about 20, about 30, about 40, about 50, about 60, about 70, about 80, about 90, or about 100 months may also be performed.

While good and poor survival outcomes may be defined relatively in comparison to each other, a “good” outcome may be viewed as a better than 50% survival rate after about 60 months post surgical intervention to remove breast cancer tumor(s). A “good” outcome may also be a better than about 60%, about 70%, about 80% or about 90% survival rate after about 60 months post surgical intervention. A “poor” outcome may be viewed as a 50% or less survival rate after about 60 months post surgical intervention to remove breast cancer tumor(s). A “poor” outcome may also be about a 70% or less survival rate after about 40 months, or about a 80% or less survival rate after about 20 months, post surgical intervention.

In one embodiment of the invention, the isolation and analysis of a breast cancer cell sample may be performed as follows:

(1) Ductal lavage or other non-invasive procedure is performed on a patient to obtain a sample.

(2) Sample is prepared and coated onto a microscope slide. Note that ductal lavage results in clusters of cells that are cytologically examined as stated above.

(3) Pathologist or image analysis software scans the sample for the presence of non-normal and/or atypical breast cancer cells.

(4) If such cells are observed, those cells are harvested (e.g. by microdissection such as LCM).

(5) RNA is extracted from the harvested cells.

(6) RNA is purified, amplified, and labeled.

(7) Labeled nucleic acid is contacted with a microarray containing polynucleotides of the genes identified herein as correlated to discriminations between breast cancer outcomes under suitable hybridization conditions, then processed and scanned to obtain a pattern of intensities of each spot (relative to a control for general gene expression in cells) which determine the level of expression of the gene(s) in the cells.

(8) The pattern of intensities is analyzed by comparison to the expression patterns of the genes in known samples of breast cancer cells correlated with outcomes (relative to the same control).

A specific example of the above method would be performing ductal lavage following a primary screen, observing and collecting non-normal and/or atypical cells for analysis. The comparison to known expression patterns, such as that made possible by a model generated by an algorithm (such as, but not limited to nearest neighbor type analysis, SVM, or neural networks) with reference gene expression data for the different breast cancer survival outcomes, identifies the cells as being correlated with subjects with good or poor outcomes. Another example would be taking a breast tumor removed from a subject after surgical intervention, isolation and preparation of breast cancer cells from the tumor for determination/identification of atypical, non-normal, or cancer cells, and isolation of said cells followed by steps 5 through 8 above.

Alternatively, the sample may permit the collection of both normal as well as cancer cells for analysis. The gene expression patterns for each of these two samples will be compared to each other as well as the model and the normal versus individual comparisons therein based upon the reference data set. This approach can be significantly more powerful that the cancer cells only approach because it utilizes significantly more information from the normal cells and the differences between normal and cancer cells (in both the sample and reference data sets) to determine the breast cancer outcome of the patient based on gene expression in the cancer cells from the sample.

With use of the present invention, skilled physicians may prescribe treatments based on prognosis determined via non-invasive samples that they would have prescribed for a patient which had previously received a diagnosis via a solid tissue biopsy.

The above discussion is also applicable where a palpable lesion is detected followed by fine needle aspiration or needle biopsy of cells from the breast. The cells are plated and reviewed by a pathologist or automated imaging system which selects cells for analysis as described above.

The present invention may also be used, however, with solid tissue biopsies. For example, a solid biopsy may be collected and prepared for visualization followed by determination of expression of one or more genes identified herein to determine the breast cancer outcome. One preferred means is by use of in situ hybridization with polynucleotide or protein identifying probe(s) for assaying expression of said gene(s).

In an alternative method, the solid tissue biopsy may be used to extract molecules followed by analysis for expression of one or more gene(s). This provides the possibility of leaving out the need for visualization and collection of only cancer cells or cells suspected of being cancerous. This method may of course be modified such that only cells that have been positively selected are collected and used to extract molecules for analysis. This would require visualization and selection as a prerequisite to gene expression analysis.

In a further modification of the above, both normal cells and cancer cells are collected and used to extract molecules for analysis of gene expression. The approach, benefits and results are as described above using non-invasive sampling.

The genes identified herein may be used to generate a model capable of predicting the breast cancer survival and recurrence outcomes of an unknown breast cell sample based on the expression of the identified genes in the sample. Such a model may be generated by any of the algorithms described herein or otherwise known in the art as well as those recognized as equivalent in the art using gene(s) (and subsets thereof) disclosed herein for the identification of breast cancer outcomes. The model provides a means for comparing expression profiles of gene(s) of the subset from the sample against the profiles of reference data used to build the model. The model can compare the sample profile against each of the reference profiles or against a model defining delineations made based upon the reference profiles. Additionally, relative values from the sample profile may be used in comparison with the model or reference profiles.

In a preferred embodiment of the invention, breast cell samples identified as normal and cancerous from the same subject may be analyzed for their expression profiles of the genes used to generate the model. This provides an advantageous means of identifying survival and recurrence outcomes based on relative differences from the expression profile of the normal sample. These differences can then be used in comparison to differences between normal and individual cancerous reference data which was also used to generate the model.

The detection of gene expression from the samples may be by use of a single microarray able to assay gene expression from some or all genes disclosed herein for convenience and accuracy.

Other uses of the present invention include providing the ability to identify breast cancer cell samples as correlated with particular breast cancer survival or recurrence outcomes for further research or study. This provides a particular advantage in many contexts requiring the identification of cells based on objective genetic or molecular criteria.

The materials for use in the methods of the present invention are ideally suited for preparation of kits produced in accordance with well known procedures. The invention thus provides kits comprising agents for the detection of expression of the disclosed genes for identifying breast cancer outcomes. Such kits optionally comprising the agent with an identifying description or label or instructions relating to their use in the methods of the present invention, is provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the present invention may also be automated in whole or in part. All aspects of the present invention may also be practiced such that they consist essentially of a subset of the disclosed genes to the exclusion of material irrelevant to the identification of breast cancer survival outcomes via a cell containing sample.

Having now generally described the invention, the same will be more readily understood through reference to the following examples which are provided by way of illustration, and are not intended to be limiting of the present invention, unless specified.

### EXAMPLES

### Example I

### General

Clinical specimen collection and clinicopathological parameters. Laser capture microdissected invasive cancer cells from a total of 124 breast cancer biopsies were used to discover two sets of genes, the expression levels of which correlate with clinical breast cancer outcomes. These genes could thus be used either individually or in combination as prognostic factors for breast cancer management. The characteristics of the 124 patient profiles in the study are shown in Table 1.

Relative expression levels of ˜22000 genes were measured from the invasive cancer cells for each of the 124 patients. Genes varying by at least 3-fold from the median expression level across the 124 patients in at least 10 patients were selected, resulting in 7090 genes.

In particular, 4 genes (DEEPEST, RACGAP1, ZNF145, MS4A7) were shown to be strong prognostic factors individually for predicting tumor recurrence after surgery and adjuvant therapies.

TABLE 1

| | Group | N | % |
|---|---|---|---|
| Age | <=45 | 30 | 24.2 |
| | 45-55 | 27 | 21.8 |
| | >55 | 67 | 54 |
| ER | positive | 66 | 53.2 |
| | negative | 58 | 46.7 |
| Node | positive | 58 | 55.2 |
| | negative | 47 | 44.8 |
| | Not avail. | 19 | 15 |
| Grade | 1 | 8 | 9.6 |
| | 2 | 29 | 34.9 |
| | 3 | 46 | 55.4 |
| | Not avail. | 41 | 33.1 |

### Example II

### Identification of ER Positive Subtypes with Different Survival Outcomes

Hierarchical clustering, based on the 7090 genes described in Example 1, of the resulting gene expression matrix (7090×124) revealed a cluster of 67-genes (the Ki67 set) the expressions of which differentiates estrogen receptor positive patients into two subgroups with distinct clinical outcomes based on overall survival over time.

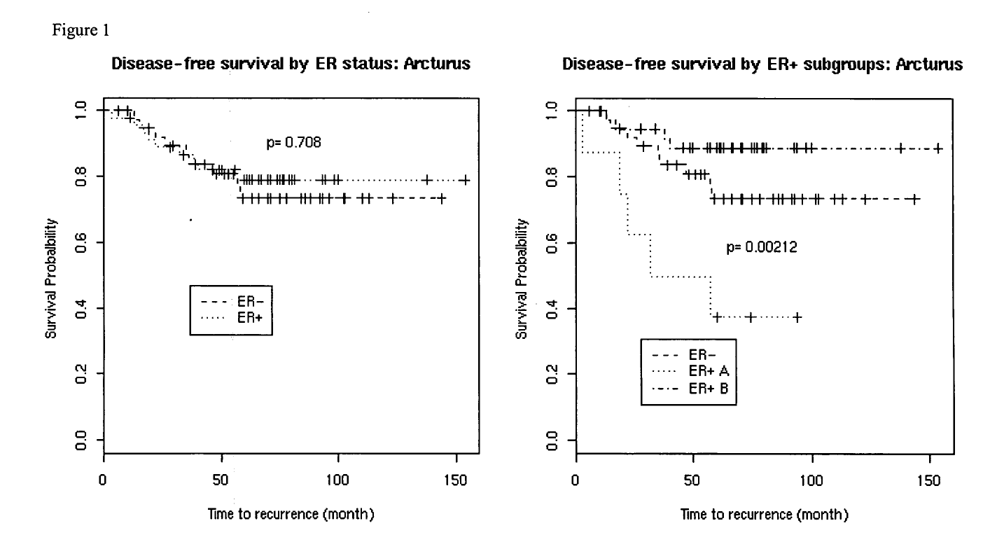

As shown in FIG. 1, left panel, a Kaplan-Meier curve on the left compares the disease-free survival of patients based on ER status, which shows slightly better survival for ER positive patients but with an insignificant p value (log-rank test). In contrast, and as shown in the right panel, when the ER positive patients are subdivided into two subgroups (A and B) based on the expression levels of the Ki67 signature genes, which are all expressed at levels above the median to define subgroup A and below the median to define subgroup B.

The three-group (ER+, subgroup A; ER+, subgroup B; and ER−) comparison shows significant differences in survival such that subgroup B subjects had significantly better survival outcomes than those of subgroup A. The ER− curve remains unchanged. This indicates that the Ki67 signature, and individual or groups of genes therein, can be used to subdivide ER positive patients into two clinically distinct subgroups based upon survival outcomes.

The identities of the genes in the Ki67 signature are shown in Table 2.

TABLE 2
Genes, the expressions of which define two ER+ subgroups

| CloneID | Gene | Description |
|---|---|---|
| 2967734 | BC007491 | EXO1 \| exonuclease 1 |
| | NM_000057 | BLM \| Bloom syndrome |
| 2849551 | AW512559 | CDC25C \| cell division cycle 25C |
| 3634656 | BC010044 | CDC20 \| CDC20 cell division cycle 20 homolog (S. cerevisiae) |
| 2961114 | BC008718 | BIRC5 \| baculoviral IAP repeat-containing 5 (survivin) |
| | NM_012112 | C20orf1 \| chromosome 20 open reading frame 1 |
| | AF399910 | DEEPEST \| mitotic spindle coiled-coil related protein |
| | NM_032997 | ZWINT \| ZW10 interactor |
| | AF331796 | HCAP-G \| chromosome condensation protein G |
| 2175265 | AI524385 | ANLN \| “anillin, actin binding protein (scraps homolog, Drosophila)” |
| 3873367 | BC010658 | KIAA0008 \| KIAA0008 gene product |
| 1338423 | AA810180 | FLJ10517 \| hypothetical protein FLJ10517 |
| | AL136794 | RACGAP1 \| Rac GTPase activating protein 1 |
| | AF334184 | FKSG42 \| FKSG42 |
| 4420248 | BC017705 | KNSL5 \| kinesin-like 5 (mitotic kinesin-like protein 1) |
| | AB035898 | KNSL7 \| kinesin-like 7 |
| 1240937 | AA714213 | ESTs, Highly similar to T47163 hypothetical protein DKFZp762E1312.1 [H. sapiens] |
| 2820741 | BC001940 | DKFZp762E1312 \| hypothetical protein DKFZp762E1312 |
| | AF017790 | HEC \| “highly expressed in cancer, rich in leucine heptad repeats” |
| 4048625 | BC013919 | TYMS \| thymidylate synthetase |
| 1699365 | AI049877 | KIAA0186 \| KIAA0186 gene product |
| 3139011 | BC001459 | RAD51 \| “RAD51 homolog (RecA homolog, E. coli) (S. cerevisiae)” |
| | AF053306 | BUB1B \| BUB1 budding uninhibited by benzimidazoles 1 homolog beta (yeast) |
| 3908972 | BC015050 | OIP5 \| Opa-interacting protein 5 |
| 1911633 | AI268609 | ESPL1 \| extra spindle poles like 1 (S. cerevisiae) |
| | NM_002417 | MKI67 \| antigen identified by monoclonal antibody Ki-67 |
| 2988318 | BC013966 | FLJ10156 \| hypothetical protein |
| 2964488 | BC013300 | STK12 \| serine/threonine kinase 12 |
| | NM_016343 | CENPF \| “centromere protein F (350/400 kD, mitosin)” |
| | AF161499 | HSPC150 \| HSPC150 protein similar to ubiquitin-conjugating enzyme |
| | AL136840 | MCM10 \| MCM10 minichromosome maintenance deficient 10 (S. cerevisiae) |
| 3028566 | BC008947 | FLJ10540 \| hypothetical protein FLJ10540 |
| 1986322 | AI273114 | ESTs, Weakly similar to I78885 serine/threonine-specific protein kinase [H. sapiens] |
| 3909951 | BC015706 | Homo sapiens, Similar to RIKEN cDNA 2810433K01 gene, clone MGC: 10200 IMAGE: 3909951, |
| | | mRNA, complete cds |
| | AF095289 | PTTG3 \| pituitary tumor-transforming 3 |
| 2262695 | AI811894 | PTTG2 \| pituitary tumor-transforming 2 |
| 1186167 | AA648922 | CDC25A \| cell division cycle 25A |
| 3138951 | BC002551 | MGC2577 \| hypothetical protein MGC2577 |
| | U74612 | FOXM1 \| forkhead box M1 |
| 3347875 | BC000703 | FLJ10468 \| hypothetical protein FLJ10468 |
| 3347571 | BC008764 | KNSL6 \| kinesin-like 6 (mitotic centromere-associated kinesin) |
| 2822981 | BC000404 | TRIP13 \| thyroid hormone receptor interactor 13 |
| 1678629 | AI082049 | ESTs |
| | NM_003504 | CDC45L \| CDC45 cell division cycle 45-like (S. cerevisiae) |
| 3345575 | BC007656 | UBE2C \| ubiquitin-conjugating enzyme E2C |
| | D84212 | STK6 \| serine/threonine kinase 6 |
| | AF011468 | STK15 \| serine/threonine kinase 15 |
| 3938081 | BC011000 | MGC16386 \| similar to RIKEN cDNA2610036L13 |
| | AF277375 | KIF4A \| kinesin family member 4A |
| 3461992 | BC000881 | CENPA \| centromere protein A (17 kD) |
| | AF108138 | PIF1 \| DMA helicase homolog PIF1 |
| | AF155827 | HELLS \| “helicase, lymphoid-specific” |
| | NM_018492 | TOPK \| T-LAK cell-originated protein kinase |
| 1686560 | AI088843 | ESTs |
| 1241465 | AA715810 | ESTs, Weakly similar to YK61_YEAST HYPOTHETICAL 39.6 KDA PROTEIN IN MTD1-NUP133 |
| | | INTERGENIC REGION [S. cerevisiae] |
| 2823731 | BC001068 | C20orf129 \| chromosome 20 open reading frame 129 |
| | AK026964 | FLJ23311 \| hypothetical protein FLJ23311 |
| 3996265 | BC005389 | LOC51053 \| geminin |
| 3901250 | BC010858 | EZH2 \| enhancer of zeste homolog 2 (Drosophila) |
| 4547136 | BC014039 | KIAA0175 \| likely ortholog of maternal embryonic leucine zipper kinase |
| 4091997 | BC017575 | CHEK1 \| CHK1 checkpoint homolog (S. pombe) |
| 669114 | AA232651 | SUV39H2 \| suppressor of variegation 3-9 (Drosophila) homolog 2; hypothetical protein FLJ23414 |
| | NM_002497 | NEK2 \| NIMA (never in mitosis gene a)-related kinase 2 |
| 4107592 | BC016330 | PIR51 \| RAD51-interacting protein |
| | AF025840 | POLE2 \| “polymerase (DMA directed), epsilon 2” |
| 3510656 | BC007633 | EIF2C2 \| “eukaryotic translation initiation factor 2C, 2” |
| | AL050151 | Homo sapiens mRNA; cDNA DKFZp586J0720 (from clone DKFZp586J0720) |

### Example III

### Molecular Signature that Correlates with the Recurrence of Breast Cancer

A molecular signature that correlates with recurrence of breast cancer after removal of cancer by surgery was identified as follows. Each of the 7090 genes from Example 1 was used to fit a univariate Cox proportional hazard regression model using the survival information available for the patients in the study. A total of 143 genes with significant p values (p<0.01) in these univariate models were selected. Hierarchical clustering of patient samples by the 143 recurrence-associated genes identified them as having expression levels that correlated with the absence or presence of breast cancer recurrence.

These 143 genes are shown in Table 3. The sign of the coefficient values in Table 3 correspond to whether a gene is positively or negatively correlated with breast cancer recurrence. A positive coefficient means that the gene is positively correlated (overexpressed) in patients with a poor (shorter) survival outcome due to recurrence and negatively correlated (underexpressed) in patients with a good or better (longer) survival outcome due to the relative absence of recurrence. A negative coefficient means that the gene is positively correlated (overexpressed) in patients with a good or better (longer) survival outcome (due to the relative absence of cancer recurrence) and negatively correlated (underexpressed) in patients with a poor (shorter) survival outcome (due to cancer recurrence).

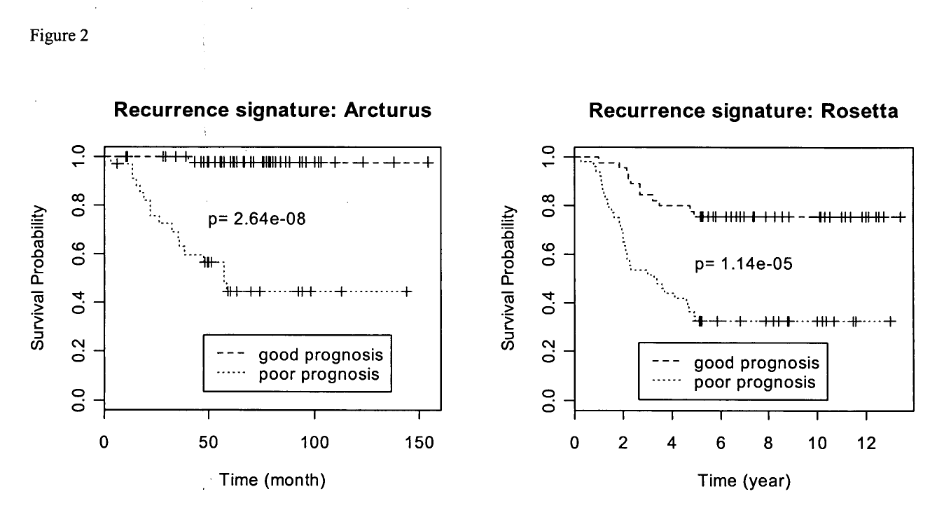

To validate this gene set, 22 of the top 27 genes from Table 3 (with the smallest p values) were mapped onto the microarray used by van't Veer et al. (Supra) via the Unigene database. The top 27 genes are provided in Table 4 while the mapping of genes are shown in Table 5 (showing identities of the genes via their GenBank ID, van't Veer et al. reference, and Unigene ID numbers). Thirteen of the 22 genes were filtered out due to low variance across the sample set, reducing the number of genes for cluster analysis to 9. The 27 gene set was used with the data from the patients of Example 1 to classify them as being in either the good prognosis or the poor prognosis group by hierarchical clustering based on disease-free survival. The results are shown in FIG. 2, left panel (Kaplan-Meier curves of patients stratified by the top 27 recurrence-associated genes).

The 9 genes not filtered out from the van't Veer et al. data were used to with the patient data therein to classify them as being in either the good prognosis or the poor prognosis group by hierarchical clustering based on disease-free survival. The results are shown in FIG. 2, right panel (Kaplan-Meier curves of patients stratified by 9 of the top 27 recurrence-associated genes).

Like FIG. 1, the horizontal axis of FIG. 2 is in time (months or years) and the vertical axis is in survival probability (where 1.0 is survival of 100% of the subjects and 0.5 is survival of 50% of the subjects). As shown in FIG. 2, differences in disease-free survival between the two groups in both datasets were highly significant.

TABLE 3
Genes, the expressions of which correlate with breast cancer recurrence

| Clone ID | gene | p | coef | desc |
|---|---|---|---|---|
| 1184567 | AA648777 | 7.58E−06 | −2.3582882 | MS4A7 \| membrane-spanning 4-domains, subfamily A, member 7 |
| 2961112 | BC005850 | 1.05E−04 | −1.845548 | CBFA2T1 \| core-binding factor, runt domain, alpha subunit 2; |
| | | | | translocated to, 1; cyclin D-related |
| 3565773 | BF432813 | 1.65E−03 | −1.2898777 | KLRB1 \| killer cell lectin-like receptor subfamily B, member 1 |
| 1352935 | AA830131 | 7.60E−03 | −1.2502516 | ZNF80 \| zinc finger protein 80 (pT17) |
| 3915193 | BC017022 | 1.80E−03 | −0.9385143 | Homo sapiens, clone MGC: 8979 IMAGE: 3915193, mRNA, complete cds |
| 2630949 | AW150267 | 2.87E−04 | −0.9172496 | C21orf9 \| chromosome 21 open reading frame 9 |
| 2714519 | AW137991 | 1.22E−03 | −0.9027559 | RELB \| v-rel reticuloendotheliosis viral oncogene homolog B, |
| | | | | nuclear factor of kappa light polypeptide gene enhancer in B-cells 3 (avian) |
| 2365891 | AI741785 | 7.56E−03 | −0.8965429 | SLIT3 \| slit homolog 3 (Drosophila) |
| | NM_006006 | 8.59E−04 | −0.8880113 | ZNF145 \| zinc finger protein 145 (Kruppel-like, expressed in promyelocytic leukemia) |
| 3645909 | BF436656 | 3.08E−03 | −0.8506381 | MFAP4 \| microfibrillar-associated protein 4 |
| 2349778 | AI806109 | 3.89E−03 | −0.8414599 | KIAA1580 \| KIAA1580 protein |
| 3504259 | BC000723 | 7.95E−03 | −0.8413126 | CRAT \| camitine acetyltransferase |
| 4342203 | BC018538 | 2.92E−04 | −0.8358585 | ALOX5AP \| arachidonate 5-lipoxygenase-activating protein |
| | AL122052 | 4.91E−04 | −0.8340079 | KIAA0793 \| KIAA0793 gene product |
| | AK025091 | 6.06E−03 | −0.8263796 | FLJ21438 \| hypothetical protein FLJ21438 |
| 2612878 | AW130888 | 2.14E−03 | −0.8099805 | PTK2B \| protein tyrosine kinase 2 beta |
| | AF244129 | 8.92E−03 | −0.807578 | LY9 \| lymphocyte antigen 9 |
| | AK027120 | 8.04E−03 | −0.8071951 | FLJ23467 \| hypothetical protein FLJ23467 |
| | AF367473 | 1.85E−04 | −0.8035463 | NYD-SP21 \| testes development-related NYD-SP21 |
| 4214447 | BC009032 | 4.52E−03 | −0.7976367 | PR48 \| protein phosphatase 2A 48 kDa regulatory subunit |
| | AB045832 | 5.30E−03 | −0.767161 | P53AIP1 \| p53-regulated apoptosis-inducing protein 1 |
| | NM_000598 | 2.64E−03 | −0.7567474 | IGFBP3 \| insulin-like growth factor binding protein 3 |
| | AI952055 | 5.06E−03 | −0.7474092 | ESTs |
| | NM_003734 | 4.42E−03 | −0.7298537 | AOC3 \| amine oxidase, copper containing 3 (vascular adhesion protein 1) |
| 4291158 | BC008392 | 6.23E−03 | −0.7260119 | UCP3 \| uncoupling protein 3 (mitochondrial, proton earner) |
| 3840457 | BC012990 | 2.07E−03 | −0.7130147 | Homo sapiens, clone IMAGE: 3840457, mRNA |
| | AB037886 | 2.20E−03 | −0.6839819 | NESH \| NESH protein |
| 3622951 | BC004300 | 6.00E−03 | −0.6820265 | VILL \| villin-like |
| | NM_015385 | 6.90E−03 | −0.6555434 | SH3D5 \| SH3-domain protein 5 (ponsin) |
| 289749 | N59284 | 4.93E−03 | −0.6497849 | ESTs |
| 3677098 | BC004864 | 2.47E−03 | −0.6450665 | PPP3CC \| protein phosphatase 3 (formerly 2B), catalytic subunit, |
| | | | | gamma isoform (calcineurin A gamma) |
| 2254324 | AI620965 | 1.08E−03 | −0.6415661 | ESTs |
| 1848897 | AI247901 | 8.54E−03 | −0.6392025 | ESTs, Weakly similar to S23650 retrovirus-related hypothetical protein II [H. sapiens] |
| 4699374 | BC017839 | 1.24E−03 | −0.6348455 | CASP4 \| caspase 4, apoptosis-related cysteine protease |
| | U90878 | 3.45E−03 | −0.6303001 | PDLIM1 \| PDZ and LIM domain 1 (elfin) |
| 2729801 | AW293849 | 3.26E−03 | −0.6188886 | ESTs, Moderately similar to I54374 gene NF2 protein [H. sapiens] |
| | AL137694 | 9.86E−03 | −0.6154861 | FLJ11286 \| hypothetical protein FLJ11286 |
| 1884362 | AI215902 | 8.92E−04 | −0.6139098 | ESTs, Highly similar to T50835 hypothetical protein [H. sapiens] |
| 3543310 | BC001609 | 7.61E−03 | −0.6113849 | WBSCR5 \| Williams-Beuren syndrome chromosome region 5 |
| 1869453 | AI264644 | 5.93E−03 | −0.6105146 | KIAA0775 \| KIAA0775 gene product |
| 3010091 | BC006107 | 1.60E−03 | −0.6088258 | ARHGAP9 \| Rho GTPase activating protein 9 |
| | NM_002405 | 5.53E−03 | −0.6041611 | MFNG \| manic fringe homolog (Drosophila) |
| | AK026343 | 1.86E−04 | −0.6018579 | FLJ22690 \| hypothetical protein FLJ22690 |
| 2227051 | AI583109 | 2.88E−03 | −0.5977938 | STAT5A \| signal transducer and activator of transcription 5A |
| 1144648 | AA613560 | 6.79E−03 | −0.5968032 | ALOX5 \| arachidonate 5-lipoxygenase |
| 206683 | H59559 | 8.44E−03 | −0.5967191 | ESTs |
| | AK021674 | 8.80E−03 | −0.5841158 | Homo sapiens cDNA FLJ11612 fis, clone HEMBA1004011 |
| 2364492 | AI741086 | 4.47E−03 | −0.5804208 | ESTs |
| | BF725007 | 8.40E−05 | −0.5750824 | ADRA2A \| adrenergic, alpha-2A-, receptor |
| | AL050391 | 3.94E−03 | −0.5724837 | Homo sapiens mRNA; cDNA DKFZp586A181 (from clone DKFZp586A181); partial cds |
| | AF367470 | 7.17E−03 | −0.5715338 | NYD-SP18 \| testes development-related NYD-SP18 |
| 293605 | AK026747 | 5.13E−03 | −0.5606859 | LOC54103 \| hypothetical protein |
| | AK025732 | 6.04E−03 | −0.5567897 | ASAH \| N-acylsphingosine amidohydrolase (acid ceramidase) |
| 1645681 | AI026838 | 4.77E−03 | −0.5536825 | ESTs, Weakly similar to NUCL_HUMAN NUCLEOLIN [H. sapiens] |
| 1670862 | AI081235 | 8.40E−03 | −0.5523028 | CD53 \| CD53 antigen |
| 3703127 | BF433686 | 5.19E−03 | −0.5433968 | Homo sapiens cDNA FLJ32651 fis, clone SYNOV2001581 |
| 1837189 | AF339781 | 4.94E−03 | −0.5432118 | GPR18 \| G protein-coupled receptor 18 |
| 4263201 | BG236645 | 1.63E−03 | −0.5426042 | ESTs |
| 4309471 | BC009956 | 3.25E−03 | −0.5390891 | HLA-DPA1 \| major histocompatibility complex, class II, DP alpha 1 |
| 31047 | R42463 | 2.11E−03 | −0.5357526 | ENTPD1 \| ectonucleoside triphosphate diphosphohydrolase 1 |
| | L02785 | 6.69E−03 | −0.5355299 | SLC26A3 \| solute carrier family 26, member 3 |
| | NM_001337 | 2.41E−03 | −0.5279489 | CX3CR1 \| chemokine (C-X3-C) receptor 1 |
| | BC016758 | 2.54E−03 | −0.5181311 | HCLS1 \| hematopoietic cell-specific Lyn substrate 1 |
| 2214761 | AI565489 | 5.26E−03 | −0.5077388 | PDE4A \| phosphodiesterase 4A, cAMP-specific (phosphodiesterase |
| | | | | E2 dunce homolog, Drosophila) |
| 2483676 | BI492073 | 4.82E−03 | −0.5068155 | ITM2A \| integral membrane protein 2A |
| 128753 | R16838 | 9.27E−03 | −0.5059294 | ESTs |
| 4548935 | BC014117 | 6.62E−03 | −0.5027082 | TBXAS1 \| thromboxane A synthase 1 (platelet, cytochrome P450, subfamily V) |
| 1734062 | AI191620 | 6.36E−03 | −0.5006683 | CDO1 \| cysteine dioxygenase, type I |
| | NM_003820 | 8.71E−03 | −0.5005279 | TNFRSF14 \| tumor necrosis factor receptor superfamily, member 14 |
| | | | | (herpesvirus entry mediator) |
| | AF305428 | 3.48E−03 | −0.4933213 | APOL1 \| apolipoprotein L, 1 |
| 3163446 | BC008734 | 6.56E−03 | −0.4928143 | FCGRT \| Fc fragment of IgG, receptor, transporter, alpha |
| 1964662 | AJ420585 | 4.18E−04 | −0.4903945 | Homo sapiens mRNA full length insert cDNA clone EUROIMAGE 1964662 |
| 1351991 | AA807346 | 5.64E−03 | −0.4893204 | Homo sapiens cDNA FLJ14296 fis, clone PLACE1008455 |
| | NM_005211 | 8.18E−03 | −0.485557 | CSF1R \| colony stimulating factor 1 receptor, formerly McDonough |
| | | | | feline sarcoma viral (v-fms) oncogene homolog |
| 2161081 | AI580271 | 9.09E−03 | −0.4836746 | AFP \| alpha-fetoprotein |
| 4862198 | BC014456 | 7.46E−03 | −0.4809022 | CHRNA6 \| cholinergic receptor, nicotinic, alpha polypeptide 6 |
| 2728733 | AW295170 | 8.18E−03 | −0.4691647 | ESTs |
| 2163996 | AI479461 | 5.00E−03 | −0.4666083 | CSR1 \| CSR1 protein |
| 3086130 | BF509235 | 4.52E−03 | −0.4567332 | KIAA1658 \| KIAA1658 protein |
| 2364383 | AI740671 | 2.10E−03 | −0.4565665 | Homo sapiens cDNA FLJ32430 fis, clone SKMUS2001129, weakly similar |
| | | | | to NAD-DEPENDENT METHANOL DEHYDROGENASE (EC 1.1.1.244) |
| 1272059 | AA743283 | 6.97E−03 | −0.4459972 | GZMK \| granzyme K (serine protease, granzyme 3; tryptase II) |
| 3902651 | BC016841 | 9.58E−03 | −0.440234 | RAB34 \| RAB34, member RAS oncogene family |
| | AB058708 | 8.44E−03 | −0.4205688 | KIAA1805 \| KIAA1805 protein |
| 40879 | R56053 | 3.88E−03 | −0.4205535 | ME3 \| malic enzyme 3, NADP(+)-dependent, mitochondrial |
| 2222621 | AI572605 | 7.12E−03 | −0.3955994 | HLA-DRA \| major histocompatibility complex, class II, DR alpha |
| 2423726 | AI860360 | 7.08E−03 | −0.3889526 | ESTs |
| 2586524 | AW080831 | 2.88E−03 | −0.3552714 | SEC14L2 \| SEC14-like 2 (S. cerevisiae) |
| 1056761 | AA574174 | 8.03E−03 | −0.3505373 | CYP2A7 \| cytochrome P450, subfamily IIA (phenobarbital-inducible), polypeptide 7 |
| | NM_033380 | 8.90E−03 | −0.3281259 | COL4A5 \| collagen, type IV, alpha 5 (Alport syndrome) |
| 2148123 | AI467846 | 8.94E−03 | −0.3273129 | IAN4L1 \| immune associated nucleotide 4 like 1 (mouse) |
| 3026606 | BE046325 | 6.30E−03 | 0.3235462 | IGFBP5 \| insulin-like growth factor binding protein 5 |
| 3939513 | BC013882 | 7.56E−03 | 0.3511008 | EYA2 \| eyes absent homolog 2 (Drosophila) |
| | AK057339 | 2.22E−03 | 0.359611 | LOC81569 \| actin like protein |
| | AF007194 | 9.59E−03 | 0.3752148 | MUC3A \| mucin 3A, intestinal |
| | AF288395 | 2.13E−03 | 0.3941496 | C1orf15 \| chromosome 1 open reading frame 15 |
| 3846346 | BC017033 | 7.82E−03 | 0.4089227 | SQLE \| squalene epoxidase |
| 3463613 | BC003684 | 5.11E−03 | 0.42608 | CXADR \| coxsackie virus and adenovirus receptor |
| 2190016 | AI538226 | 8.41E−04 | 0.4327835 | GNG4 \| guanine nucleotide binding protein 4 |
| 2138200 | AI522215 | 8.62E−03 | 0.4352044 | KIAA1804 \| KIAA1804 protein |
| 3087716 | BF510979 | 7.81E−03 | 0.4409617 | DHDH \| dihydrodiol dehydrogenase (dimeric) |
| 5677199 | BM129393 | 7.66E−03 | 0.4526335 | GDF1 \| growth differentiation factor 1 |
| 2144913 | AI452634 | 4.23E−03 | 0.4561948 | GPR64 \| G protein-coupled receptor 64 |
| | M95585 | 3.62E−03 | 0.5164108 | HLF \| hepatic leukemia factor |
| 3932186 | BC005345 | 6.63E−03 | 0.5470575 | GTF2H2 \| general transcription factor IIH, polypeptide 2 (44 kD subunit) |
| 2968940 | AW613854 | 7.48E−03 | 0.5487125 | ESTs, Moderately similar to S02826 nonhistone chromosomal protein HMG-1 [H. sapiens] |
| | AF017790 | 5.02E−03 | 0.5639359 | HEC \| highly expressed in cancer, rich in leucine heptad repeats |
| | AY049737 | 6.54E−03 | 0.5703928 | NPM3 \| nucleophosmin/nucleoplasmin, 3 |
| | U87791 | 8.91E−03 | 0.5755378 | HBS1L \| HBS1-like (S. cerevisiae) |
| 3915484 | BC017053 | 9.97E−03 | 0.5772075 | ACOX3 \| acyl-Coenzyme A oxidase 3, pristanoyl |
| | AF100751 | 9.93E−03 | 0.5901307 | LOC51661 \| FK506-binding protein |
| | AF206673 | 9.98E−03 | 0.6026123 | BRF2 \| BRF2, subunit of RNA polymerase III transcription initiation factor, BRF1-like |
| 1251833 | AA731207 | 6.11E−03 | 0.6062738 | FLJ10858 \| hypothetical protein FLJ10858 |
| 2507739 | AI961369 | 2.77E−03 | 0.6190266 | INSIG1 \| insulin induced gene 1 |
| 3504930 | BC005141 | 5.35E−03 | 0.621928 | GALK2 \| galactokinase 2 |
| | AL136570 | 6.98E−03 | 0.6225449 | LHX6 \| LIM homeobox protein 6 |
| 2735278 | AW450731 | 7.74E−03 | 0.6254488 | FLJ14642 \| hypothetical protein FLJ14642 |
| | AK025820 | 7.09E−03 | 0.6282847 | FLJ22167 \| hypothetical protein FLJ22167 |
| 2975886 | AW629176 | 9.47E−04 | 0.6387345 | ESTs, Weakly similar to I38022 hypothetical protein [H. sapiens] |
| | AF116670 | 4.34E−03 | 0.6509022 | NP \| nucleoside phosphorylase |
| 1630968 | AI018605 | 3.48E−03 | 0.6689059 | ESTs |
| 3996449 | BC015107 | 7.48E−03 | 0.6910834 | FLJ13433 \| hypothetical protein FLJ13433 |
| | AB049113 | 9.76E−03 | 0.7010916 | DUT \| dUTP pyrophosphatase |
| | AK025543 | 8.56E−03 | 0.707967 | KIAA1345 \| KIAA1345 protein |
| | AF053306 | 6.31E−03 | 0.7227021 | BUB1B \| BUB1 budding uninhibited by benzimidazoles 1 homolog beta (yeast) |
| 3010092 | BC008954 | 9.78E−03 | 0.7272841 | SLC29A1 \| solute carrier family 29 (nucleoside transporters), member 1 |
| | NM_001685 | 6.48E−03 | 0.7348505 | ATP5J \| ATP synthase, H+ transporting, mitochondrial F0 complex, subunit F6 |
| 4838878 | BC016751 | 5.11E−03 | 0.7473835 | PCDHB3 \| protocadherin beta 3 |
| 2175265 | AI524385 | 2.93E−03 | 0.7701449 | ANLN \| anillin, actin binding protein (scraps homolog, Drosophila) |
| 3010727 | BE206076 | 1.25E−04 | 0.7737357 | ALK \| anaplastic lymphoma kinase (Ki-1) |
| | X62534 | 4.09E−03 | 0.7848284 | HMG2 \| high-mobility group (nonhistone chromosomal) protein 2 |
| | NM_018669 | 3.59E−03 | 0.8006609 | WDR4 \| WD repeat domain 4 |
| | AB035898 | 3.72E−03 | 0.8063743 | KNSL7 \| kinesin-like 7 |
| | NM_006734 | 8.82E−03 | 0.8275957 | HIVEP2 \| human immunodeficiency virus type I enhancer binding protein 2 |
| | AF399910 | 1.43E−03 | 0.8370408 | DEEPEST \| mitotic spindle coiled-coil related protein |
| | AF331796 | 2.69E−03 | 0.8636081 | HCAP-G \| chromosome condensation protein G |
| | AF073518 | 6.93E−03 | 0.8902775 | SERF1A \| small EDRK-rich factor 1A (telomeric) |
| 1870184 | AI245807 | 1.20E−03 | 0.8964932 | MGC14798 \| similar to RIKEN cDNA 5730421E18 gene |
| 4509200 | BC012919 | 1.19E−03 | 0.9209921 | KLF7 \| Kruppel-like factor 7 (ubiquitous) |
| 3926227 | BC009855 | 1.51E−03 | 0.9417395 | FLJ14909 \| hypothetical protein FLJ14909 |
| 1337864 | AA811376 | 2.04E−03 | 1.0013265 | FLJ10545 \| hypothetical protein FLJ10545 |
| 4109322 | BC016782 | 2.22E−03 | 1.0106451 | KIAA0101 \| KIAA0101 gene product |
| | AF334184 | 1.66E−04 | 1.0332632 | FKSG42 \| FKSG42 |
| | AL136794 | 1.98E−04 | 1.1631056 | RACGAP1 \| Rac GTPase activating protein 1 |

TABLE 4
Top 27 genes

| gene | p | coef | desc |
|---|---|---|---|
| BE206076 | 7.27446E−05 | 1.2270555 | ALK \| anaplastic lymphoma kinase (Ki-1) |
| AF040628 | 0.000116979 | 0.9344455 | ED1 \| ectodermal dysplasia 1, anhidrotic |
| BF725007 | 0.000331932 | −0.5801744 | ADRA2A \| adrenergic, alpha-2A-, receptor |
| AF367473 | 0.00068377 | −0.8560356 | NYD-SP21 \| testes development-related NYD-SP21 |
| AI245807 | 0.000789859 | 1.0889127 | MGC14798 \| similar to RIKEN cDNA 5730421E18 gene |
| AI215902 | 0.00086535 | −0.6485096 | ESTs, Highly similar to T50835 hypothetical protein [H. sapiens] |
| AF334184 | 0.000883849 | 1.0194484 | FKSG42 \| FKSG42 |
| AW137991 | 0.000969904 | −1.1497247 | RELB \| v-rel reticuloendotheliosis viral oncogene homolog B, |
| | | | nuclear factor of kappa light polypeptide gene enhancer in B-cells 3 (avian) |
| AA648777 | 0.001013976 | −1.6577293 | MS4A7 \| membrane-spanning 4-domains, subfamily A, member 7 |
| BC017053 | 0.001247656 | 0.8780177 | ACOX3 \| acyl-Coenzyme A oxidase 3, pristanoyl |
| AL136570 | 0.00133504 | 0.822904 | LHX6 \| LIM homeobox protein 6 |
| AL136794 | 0.001396755 | 1.1506902 | RACGAP1 \| Rac GTPase activating protein 1 |
| AF331796 | 0.001490968 | 1.2361318 | HCAP-G \| chromosome condensation protein G |
| BC005850 | 0.001567107 | −1.5082099 | CBFA2T1 \| core-binding factor, runt domain, alpha subunit 2; translocated to, 1; |
| | | | cyclin D-related |
| AF399910 | 0.00160607 | 0.8764045 | DEEPEST \| mitotic spindle coiled-coil related protein |
| AK057339 | 0.001660726 | 0.4461839 | LOC81569 \| actin like protein |
| NM_003265 | 0.001664793 | −0.7178243 | TLR3 \| toll-like receptor 3 |
| AK026343 | 0.001697437 | −0.5688145 | FLJ22690 \| hypothetical protein FLJ22690 |
| BC018538 | 0.001728603 | −0.7262933 | ALOX5AP \| arachidonate 5-lipoxygenase-activating protein |
| AI806109 | 0.001789313 | −1.0762434 | KIAA1580 \| KIAA1580 protein |
| AL122052 | 0.001810196 | −0.9558644 | KIAA0793 \| KIAA0793 gene product |
| BC012919 | 0.002008228 | 0.9606709 | KLF7 \| Kruppel-like factor 7 (ubiquitous) |
| BC008392 | 0.002082612 | −1.0314954 | UCP3 \| uncoupling protein 3 (mitochondrial, proton carrier) |
| BF432813 | 0.002110755 | −1.2468785 | KLRB1 \| killer cell lectin-like receptor subfamily B, member 1 |
| AI741086 | 0.002281532 | −0.675948 | ESTs |
| AK022729 | 0.002327927 | −0.9643334 | KIAA1681 \| KIAA1681 protein |
| NM_006006 | 0.002390747 | −1.0628839 | ZNF145 \| zinc finger protein 145 (Kruppel-like, expressed in promyelocytic leukemia) |

TABLE 5

| GenBank | van't Veer et al. | UniGene |
|---|---|---|
| AA648777 | AF201951 | Hs.11090 |
| AF367473 | AL137391 | Hs.28514 |
| AK022729 | Contig30485_RC | Hs.42656 |
| AI741086 | Contig39054_RC | Hs.115122 |
| AK022729 | Contig47136_RC | Hs.42656 |
| AI215902 | Contig52342_RC | Hs.88845 |
| BF725007 | Contig53357_RC | Hs.249159 |
| BF725007 | NM_000681 | Hs.249159 |
| AF040628 | NM_001399 | Hs.105407 |
| BC018538 | NM_001629 | HS.100194 |
| BF432813 | NM_002258 | Hs.169824 |
| NM_003265 | NM_003265 | Hs.29499 |
| BC008392 | NM_003356 | Hs.101337 |
| BC017053 | NM_003501 | HS.12773 |
| BC012919 | NM_003709 | Hs.21599 |
| BE206076 | NM_004304 | Hs.278572 |
| BC005850 | NM_004349 | Hs.31551 |
| NM_006006 | NM_006006 | Hs.37096 |
| AF399910 | NM_006461 | Hs.16244 |
| AW137991 | NM_006509 | Hs.858 |
| AL136794 | NM_013277 | Hs.23900 |
| AL136570 | NM_014368 | Hs.103137 |
| AL122052 | NM_014808 | Hs.301283 |
| AK057339 | U20582 | Hs.2149 |

### Example IV

### Individual Genes that are Expressed at Higher than Median Levels and Correlated with the Recurrence of Breast Cancer

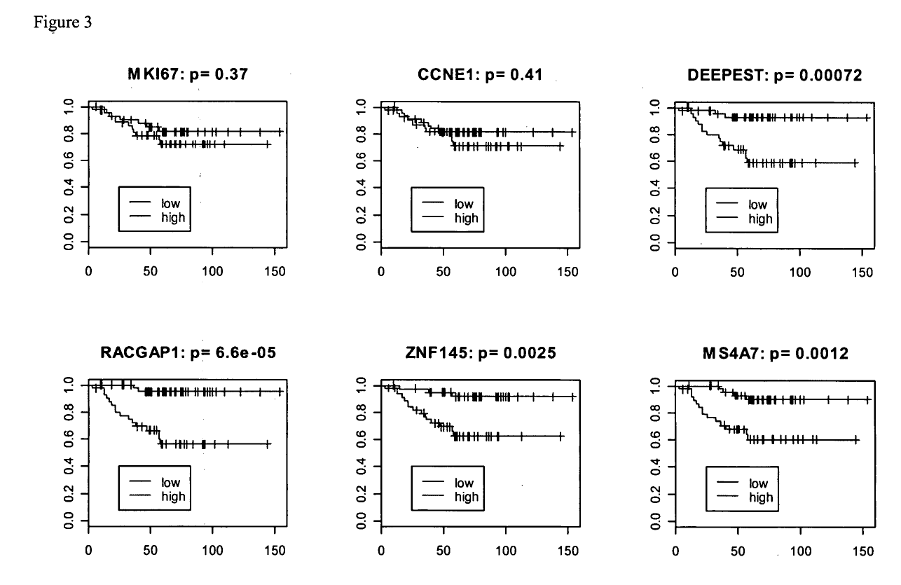

DEEPEST, RACGAP1, ZNF145 and MS4A7 were found to each be significantly associated with tumor recurrence. In both the datasets used in FIG. 2, patients were divided into high and low expression groups relative to the overall median for each gene across all patients, and their survival curves were compared (see FIG. 3, which shows Kaplan-Meier disease-free survival curves). The first six graphs in FIG. 3 display the results using the dataset from the 124 patients of Example 1; the X-axis is in months. The second six graphs in FIG. 3 display the results using the dataset from van't Veer et al. with the X-axis in years. The Y-axis for both are “survival probability” as described above. As control, MKI67 and CCNE1, two genes known to be associated with aggressive cancers were analyzed in the same manner.

### Example V

### Correlation with Tumor Grade

The expression pattern of the Ki67 genes was also found to be strongly correlated with tumor grade. All 67 genes were found to be relatively overexpressed in subjects with high-grade (grade 3) tumors and underexpressed in subjects with low-grade (grades 1 and 2) tumors.

### Example VI

### Cross-Validation Based on Recurrence Gene Signatures

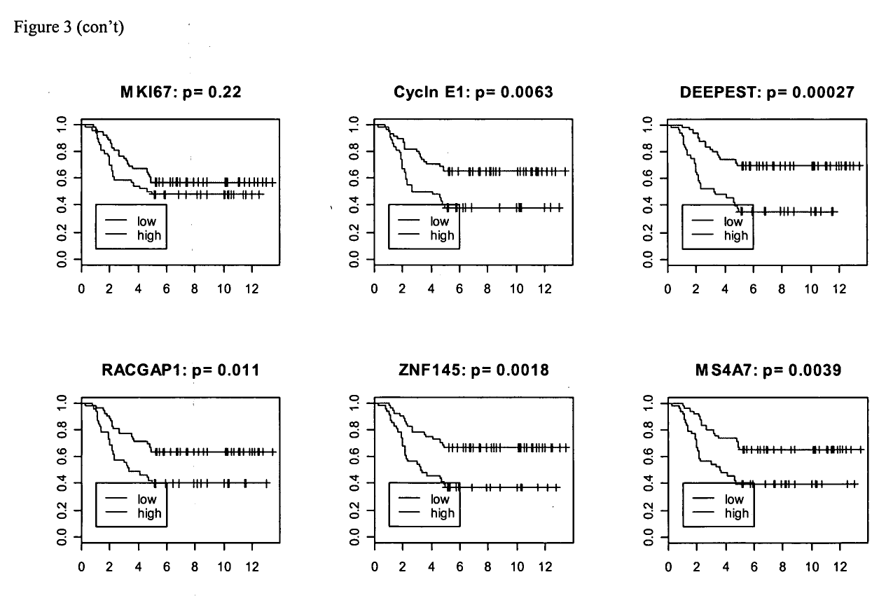

As shown in FIG. 4, eighty-five selected EP positive (ERP) samples (training dataset) were evaluated for survival probability based upon 141 recurrence gene signatures. The horizontal axis of FIG. 4 is in time (months) and vertical axis is in survival probability.

Table 6 lists the 141 recurrence-associated genes. The sign of the coefficient values in Table 6 corresponds to whether a gene is positively or negatively correlated with breast cancer recurrence. A positive coefficient (score >0) means that the gene is positively correlated in patients with a poor (shorter) survival outcome and negatively correlated coefficients (score <0) mean that the gene is correlated in patients with better (longer) survival outcomes.

The 141 genes were identified from a starting gene pool of 180 genes, wherein the 141 genes had expression levels that correlated with the absence or presence of breast cancer recurrence.

TABLE 6
Genes, the expression of which correlate with breast cancer recurrence.

| gene | p | coef | desc |
|---|---|---|---|
| AK026216 | 0.0000124 | −0.5776051 | Homo sapiens cDNA: FLJ22563 fis, clone HSI01928 |
| BC012889 | 0.0000157 | 0.4919877 | APLP1 \| amyloid beta (A4) precursor-like protein 1 |
| N30158 | 0.0000207 | −0.5425882 | ESTs |
| AL109775 | 0.0000579 | 0.3522512 | SH3GL3 \| SH3-domain GRB2-like 3 |
| AJ275978 | 0.0000778 | 0.5278802 | CTAG1 \| cancer/testis antigen 1 |
| AI340191 | 0.000156 | −0.616326 | HSPC072 \| HSPC072 protein |
| AJ409065 | 0.000171 | −0.564874 | LEAP-2 \| liver-expressed antimicrobial peptide 2 |
| AL110279 | 0.000201 | −0.5886589 | H-L(3)MBT \| lethal (3) malignant brain tumor l(3)mbt protein (Drosophila) homolog |
| AF057164 | 0.000345 | −0.6014184 | SLC22A5 \| solute carrier family 22 (organic cation transporter), member 5 |
| AA398715 | 0.00035 | −0.550304 | Homo sapiens cDNA FLJ11529 fis, clone HEMBA1002629 |
| AI609043 | 0.000363 | 0.3483688 | ESTs, Highly similar to T50606 hypothetical protein DKFZp761J107.1 [H. sapiens] |
| AI277016 | 0.000367 | −0.4165628 | ESTs |
| AK024715 | 0.000376 | −0.5755967 | FLJ21062 \| hypothetical protein FLJ21062 |
| AL117396 | 0.000385 | −0.5432668 | DKFZP586M0622 \| DKFZP586M0622 protein |
| AF277290 | 0.000465 | 0.4119716 | LOC81501 \| DC-specific transmembrane protein |
| AW294857 | 0.000543 | −0.5054436 | LOC51161 \| g20 protein |
| AA845338 | 0.000684 | −0.3511727 | FMO5 \| flavin containing monooxygenase 5 |
| NM_003842 | 0.00074 | −0.5120187 | TNFRSF10B \| tumor necrosis factor receptor superfamily, member 10b |
| AI400402 | 0.000754 | 0.4795643 | GBP2 \| guanylate binding protein 2, interferon-inducible |
| AF035281 | 0.00077 | −0.8496583 | Homo sapiens clone 23903 mRNA sequence |
| AK026215 | 0.000794 | −0.4874715 | Homo sapiens cDNA: FLJ22562 fis, clone HSI01814 |
| BC000715 | 0.000851 | 0.3567116 | CLECSF9 \| C-type (calcium dependent, carbohydrate-recognition domain) lectin, superfamily member 9 |
| AI740671 | 0.00095 | −0.3584972 | Homo sapiens cDNA FLJ32430 fis, clone SKMUS2001129, weakly similar to NAD-DEPENDENT |
| | | | METHANOL DEHYDROGENASE (EC 1.1.1.244) |
| AK024991 | 0.00099 | −0.5133878 | TRIP8 \| thyroid hormone receptor interactor 8 |
| AL136790 | 0.00101 | −0.5095425 | DKFZp434F1819 \| hypothetical protein DKFZp434F1819 |
| BC006000 | 0.00107 | 0.3974168 | MGC12536 \| hypothetical protein MGC12536 |
| AI139409 | 0.00111 | 0.5442699 | CDKN1C \| cyclin-dependent kinase inhibitor 1C (p57, Kip2) |
| AA738043 | 0.00134 | −0.3124779 | SCAP1 \| src family associated phosphoprotein 1 |
| AF055634 | 0.00145 | 0.3174865 | UNC5C \| unc-5 homolog B (C. elegans) |
| AL136721 | 0.00146 | −0.5008718 | DKFZP566K1946 \| hypothetical protein DKFZp566K1946 |
| U62662 | 0.00154 | 0.3392979 | CHIT1 \| chitinase 1 (chitotriosidase) |
| BC012381 | 0.00173 | 0.3432002 | FLJ10430 \| hypothetical protein FLJ10430 |
| AW195539 | 0.00178 | −0.4498633 | DDO \| D-aspartate oxidase |
| AI240072 | 0.00187 | −0.4635729 | Homo sapiens mRNA full length insert cDNA clone EUROIMAGE 2344436 |
| BC000544 | 0.00196 | 0.2768966 | KCNJ8 \| potassium inwardly-rectifying channel, subfamily J, member 8 |
| AA029452 | 0.00196 | −0.4884253 | KCNQ1OT1 \| KCNQ1 overlapping transcript 1 |
| BC005939 | 0.00225 | 0.4954123 | PTGDS \| prostaglandin D2 synthase (21 kD, brain) |
| BC000748 | 0.00226 | 0.3983599 | TUBB4 \| tubulin, beta, 4 |
| BC008632 | 0.00228 | 0.3394714 | Homo sapiens, clone IMAGE: 3464195, mRNA |
| AI922204 | 0.00246 | −0.2125933 | Homo sapiens cDNA FLJ30298 fis, clone BRACE2003172 |
| R55764 | 0.00247 | −0.5712032 | Homo sapiens cDNA FLJ33034 fis, clone THYMU2000236 |
| BF477905 | 0.00254 | −0.4507438 | ESTs |
| BC005816 | 0.00255 | 0.3445679 | DTX1 \| deltex homolog 1 (Drosophila) |
| R56053 | 0.00267 | −0.303724 | ME3 \| malic enzyme 3, NADP(+)-dependent, mitochondrial |
| AK027191 | 0.0027 | −0.4094706 | Homo sapiens cDNA: FLJ23538 fis, clone LNG08010, highly similar to BETA2 Human MEN1 |
| | | | region clone epsilon/beta mRNA |
| AB028641 | 0.00309 | 0.241634 | SOX11 \| SRY (sex determining region Y)-box 11 |
| AK027251 | 0.0032 | −0.3436063 | FLJ23598 \| hypothetical protein FLJ23598 |
| AF153330 | 0.0032 | −0.3901168 | SLC19A2 \| solute carrier family 19 (thiamine transporter), member 2 |
| AK056720 | 0.0032 | 0.442301 | Homo sapiens cDNA FLJ32158 fis, clone PLACE6000231 |
| BE671445 | 0.00323 | −0.5541909 | ESTs |
| NM_007023 | 0.00323 | −0.383125 | CAMP-GEFII \| cAMP-regulated guanine nucleotide exchange factor II |
| BC007045 | 0.0033 | 0.4541716 | MLF1 \| myeloid leukemia factor 1 |
| AY007114 | 0.00335 | −0.4164539 | Homo sapiens clone TCCCTA00151 mRNA sequence |
| AB033110 | 0.00335 | −0.4816304 | KIAA1284 \| KIAA1284 protein |
| AI126271 | 0.00381 | −0.3325846 | Homo sapiens cDNA FLJ31235 fis, clone KIDNE2004681, moderately similar to Mus musculus |
| | | | peroxisomal long chain acyl-CoA thioesterase Ib (Pte1b) gene |
| U66046 | 0.00396 | −0.3632168 | Homo sapiens clone 161455 breast expressed mRNA from chromosome X |
| BC017422 | 0.004 | −0.4540171 | Homo sapiens, clone MGC: 27375 IMAGE: 4688423, mRNA, complete cds |
| M76558 | 0.00411 | −0.3517135 | CACNA1D \| calcium channel, voltage-dependent, L type, alpha 1D subunit |
| AA627358 | 0.00412 | −0.2554568 | ESTs |
| AF237813 | 0.00415 | −0.3188455 | NPD009 \| NPD009 protein |
| BE501103 | 0.00415 | −0.5697021 | Homo sapiens cDNA FLJ32173 fis, clone PLACE6000953 |
| BC005362 | 0.0042 | −0.4013912 | ARHI \| ras homolog gene family, member I |
| BC010563 | 0.00427 | −0.3934675 | Homo sapiens, clone MGC: 18111 IMAGE: 4152811, mRNA, complete cds |
| AB028140 | 0.00446 | −0.5082397 | TMPRSS5 \| transmembrane protease, serine 5 (spinesin) |
| BC002668 | 0.00446 | −0.3559138 | PECI \| peroxisomal D3,D2-enoyl-CoA isomerase |
| BC005948 | 0.00448 | 0.2903151 | SMPX \| small muscle protein, X-linked |
| AI382972 | 0.00458 | −0.5293407 | TPBG \| trophoblast glycoprotein |
| BE551149 | 0.00459 | −0.4024775 | ESTs |
| AK024893 | 0.00459 | 0.3693678 | FLJ21240 \| hypothetical protein FLJ21240 |
| AJ420490 | 0.00461 | −0.2242605 | IL20RA \| interleukin 20 receptor, alpha |
| AF261655 | 0.00479 | −0.3113893 | HMIC \| 1,2-alpha-mannosidase IC |
| AF285089 | 0.00493 | −0.5135262 | LLT1 \| lectin-like NK cell receptor |
| AK000520 | 0.0051 | 0.2643999 | FLJ20513 \| hypothetical protein FLJ20513 |
| AF132197 | 0.00517 | −0.4538855 | PRO1331 \| hypothetical protein PRO1331 |
| AW139156 | 0.00543 | 0.4096147 | CRMP5 \| collapsin response mediator protein-5; CRMP3-associated molecule |
| U62325 | 0.00561 | −0.5801082 | APBB2 \| amyloid beta (A4) precursor protein-binding, family B, member 2 (Fe65-like) |
| AK026740 | 0.00581 | 0.4834703 | Homo sapiens cDNA: FLJ23087 fis, clone LNG06994, highly similar to AF161368 |
| | | | Homo sapiens HSPC105 mRNA |
| AA985520 | 0.00583 | −0.5217466 | ESTs |
| AA633845 | 0.00626 | −0.3464769 | ESTs |
| NM_014298 | 0.00628 | 0.3378903 | QPRT \| quinolinate phosphoribosyltransferase (nicotinate-nucleotide pyrophosphorylase (carboxylating)) |
| AF208111 | 0.00638 | −0.3116422 | IL17BR \| interleukin 17B receptor |
| BC017733 | 0.00641 | 0.4047293 | MRAS \| muscle RAS oncogene homolog |
| AF222340 | 0.00649 | −0.4462726 | ARTS-1 \| type 1 tumor necrosis factor receptor shedding aminopeptidase regulator |
| NM_005430 | 0.00653 | 0.3978767 | WNT1 \| wingless-type MMTV integration site family, member 1 |
| NM_002281 | 0.00669 | 0.319678 | KRTHB1 \| keratin, hair, basic, 1 |
| AF285109 | 0.00671 | 0.31544 | 3-Sep \| septin 3 |
| BC018537 | 0.00683 | −0.4326731 | Homo sapiens, Similar to RIKEN cDNA 1300003P13 gene, clone MGC: 16810 IMAGE: 4340152, mRNA, |
| | | | complete cds |
| NM_004430 | 0.00685 | −0.3053692 | EGR3 \| early growth response 3 |
| AA584306 | 0.00685 | −0.5508093 | GALNT5 \| UDP-N-acetyl-alpha-D-galactosamine: polypeptide N-acetylgalactosaminyltransferase 5 |
| | | | (GalNAc-T5) |
| BC012198 | 0.00686 | 0.394402 | Homo sapiens, clone MGC: 4408 IMAGE: 2906200, mRNA, complete cds |
| AI572737 | 0.00702 | −0.4298417 | ESTs |
| AI950985 | 0.00726 | −0.3315239 | ESTs |
| AI829793 | 0.00734 | −0.5945521 | EST |
| X51420 | 0.00735 | 0.261491 | TYRP1 \| tyrosinase-related protein 1 |
| AF067223 | 0.00747 | 0.4465152 | PDE9A \| phosphodiesterase 9A |
| AF130068 | 0.00766 | −0.4029209 | SERPINA1 \| serine (or cysteine) proteinase inhibitor, clade A (alpha-1 |
| | | | antiproteinase, antitrypsin), member 1 |
| NM_003508 | 0.00769 | 0.2270396 | FZD9 \| frizzled homolog 9 (Drosophila) |
| AK000397 | 0.00775 | 0.3705402 | FLJ10351 \| likely ortholog of mouse piwi like homolog 1 (Drosophila)-like |
| AA513133 | 0.00778 | −0.4115009 | ESTs, Weakly similar to 1209280A tropomyosin [H. sapiens] |
| AA811922 | 0.00819 | 0.4471232 | FLJ10140 \| hypothetical protein FLJ10140 |
| AL390129 | 0.00823 | 0.2753104 | ATP8A2 \| ATPase, aminophospholipid transporter-like, Class I, type 8A, member 2 |
| BC014336 | 0.00846 | −0.3339104 | HHEX \| hematopoietically expressed homeobox |
| AJ000534 | 0.00876 | 0.2307116 | SGCE \| sarcoglycan, epsilon |
| AW444896 | 0.00891 | 0.2076823 | ESTs |
| AL136789 | 0.00893 | −0.4066268 | DKFZp434F1719 \| hypothetical protein DKFZp434F1719 |
| AF207664 | 0.00894 | 0.2878826 | ADAMTS1 \| a disintegrin-like and metalloprotease (reprolysin type) with thrombospondin type 1 motif, 1 |
| AF016267 | 0.00904 | −0.4665036 | TNFRSF10C \| tumor necrosis factor receptor superfamily, member 10c, decoy without an intracellular domain |
| AA805317 | 0.00906 | −0.2905281 | HOXC4 \| homeo box C4 |
| NM_032961 | 0.00909 | 0.4222674 | PCDH10 \| protocadherin 10 |
| BC015706 | 0.00909 | 0.4310617 | Homo sapiens, Similar to RIKEN cDNA 2810433K01 gene, clone MGC: 10200 IMAGE: 3909951, mRNA, |
| | | | complete cds |
| AF112345 | 0.00917 | −0.4117301 | ITGA10 \| integrin, alpha 10 |
| AI651102 | 0.00918 | −0.361676 | ESTs |
| BC001492 | 0.00954 | 0.2916312 | CNTFR \| ciliary neurotrophic factor receptor |
| BC014189 | 0.00961 | −0.2332876 | MGC20702 \| hypothetical protein MGC20702 |
| AA890146 | 0.00964 | 0.486204 | ESTs |
| AI459134 | 0.00969 | 0.310778 | CD163\|CD163 antigen |
| D13626 | 0.00969 | 0.2913298 | GPR105 \| G protein-coupled receptor 105 |
| AW015171 | 0.0098 | −0.3731535 | KIAA0022 \| KIAA0022 gene product |
| BC007631 | 0.00985 | 0.3549781 | MGC15827 \| hypothetical protein MGC15827 |
| AF070587 | 0.00988 | −0.3138695 | Homo sapiens clone 24741 mRNA sequence |
| BC011628 | 0.00999 | −0.3693683 | EPHX2 \| epoxide hydrolase 2, cytoplasmic |
| BF111883 | 0.0104 | 0.3647822 | bA430M15.1 \| novel protein (ortholog of rat four repeat ion channel) |
| AF011333 | 0.0105 | −0.3938612 | LY75 \| lymphocyte antigen 75 |
| AB042410 | 0.0106 | 0.2578638 | GPR88 \| G-protein coupled receptor 88 |
| AB012643 | 0.0106 | 0.4935166 | ALPL \| alkaline phosphatase, liver/bone/kidney |
| BC000558 | 0.0109 | −0.2313315 | MAPT \| microtubule-assotiated protein tau |
| M81883 | 0.011 | −0.3791012 | GAD1 \| glutamate decarboxylase 1 (brain, 67 kD) |
| AA765256 | 0.0112 | −0.3259152 | ESTs, Weakly similar to unnamed protein product [H. sapiens] |
| AL353948 | 0.0113 | 0.3592147 | Homo sapiens mRNA; cDNA DKFZp761P0114 (from clone DKFZp761P0114) |
| AI686160 | 0.0113 | −0.3480712 | ESTs |
| BF112017 | 0.0113 | 0.2891108 | KCNE1L \| potassium voltage-gated channel, Isk-related family, member 1-like |
| AI393476 | 0.0114 | 0.3087115 | RBP1 \| retinol binding protein 1 , cellular |
| R41429 | 0.0115 | −0.3424425 | ATP7B \| ATPase, Cu++ transporting, beta polypeptide (Wilson disease) |
| L20433 | 0.0117 | 0.4816472 | POU4F1 \| POU domain, class 4, transcription factor 1 |
| BC001847 | 0.0117 | 0.3254246 | MGC4504 \| hypothetical protein MGC4504 |
| AK025672 | 0.0118 | −0.4356503 | FLJ20761 \| hypothetical protein FLJ20761 |
| BC014794 | 0.0118 | −0.3526159 | FLJ20574 \| hypothetical protein FLJ20574 |
| BC002415 | 0.012 | −0.2664004 | GSTT2 \| glutathione S-transferase theta 2 |
| AK027179 | 0.012 | −0.4551677 | ATF7 \| activating transcription factor 7 |
| AL353944 | 0.0124 | −0.3365367 | Homo sapiens mRNA; cDNA DKFZp761J1112 (from clone DKFZp761J1112) |
| NM_012173 | 0.0125 | −0.4651839 | FBXO25 \| F-box only protein 25 |

## NOTE.
P and coef are derived from Cox proportional hazard models. Genes with positive coefs are expressed higher in recurrence group, and those with negative coefs are expressed higher in no-recurrence group

### Example VII

### Prognosis of Recurrence Utilizing the 141 Recurrence Signature Genes

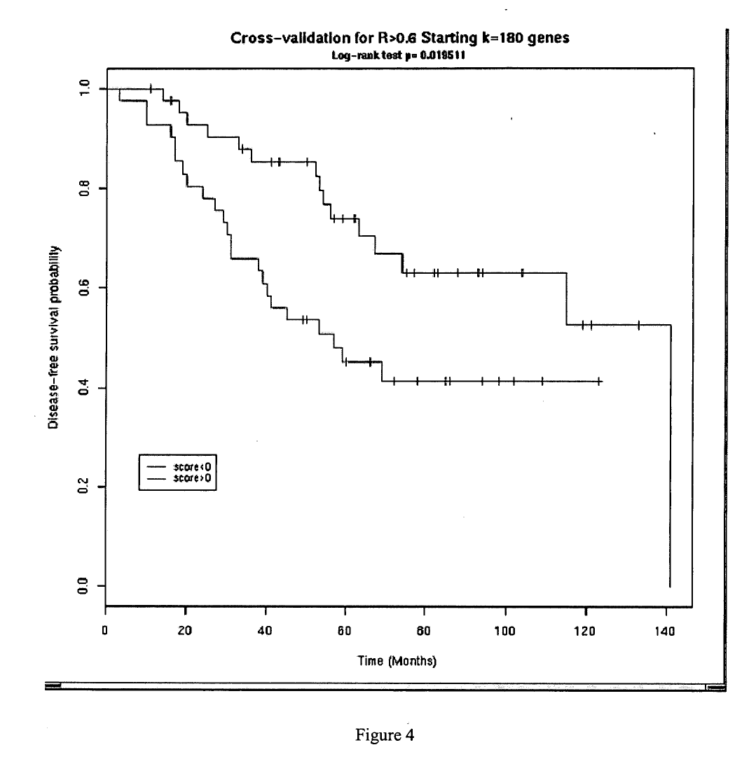

Sixty-six patients having ERP breast cancer (test dataset) were evaluated utilizing the identified 141 signature genes in order to predict survival outcomes, based upon recurrence of the breast cancer. The prognostic results are shown in FIG. 5.

Another group of patients were evaluated; this group contained both ERP and ER negative (ERN) patients, wherein the total number of patients evaluated was 162 (test dataset). The prognostic results for this second group of patients also are shown in FIG. 5.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. (canceled)

2. (canceled)

3. (canceled)

4. (canceled)

5. A method to determine the survival outcome of a breast cancer afflicted subject or determine prognosis of a subject having breast cancer, said method comprising assaying a sample of breast cancer cells of said subject for the expression level(s) of one or more genes listed in Table 2, 3, 4, and/or 6.

6. (canceled)

7. A method of determining the prognosis of a subject having breast cancer correlated with the over or under expression of one or more genes in Table 2, 3, 4, and/or 6 said method comprising assaying for the expression level(s) of said one or more genes in a breast cancer cell from said subject.

8. The method of claim 5 wherein said assaying comprises preparing RNA from said sample.

9. The method of claim 8 wherein said RNA is used for quantitative PCR.

10. The method of claim 5 wherein said assaying comprises using an array.

11. The method of claim 5 wherein said sample is a ductal lavage or fine needle aspiration sample.

12. The method of claim 11 wherein said sample is microdissected to isolate one or more cells suspected of being breast cancer cells.

13. The method of claim 5 wherein said assaying comprises preparing RNA from said sample and optionally using said RNA for quantitative PCR.

14. The method of claim 8 wherein said assaying comprises using an array.

15. The method of claim 5 wherein said sample is a ductal lavage or fine needle aspiration sample, which sample is optionally microdissected to isolate one or more cells suspected of being breast cancer cells.

16. The method of claim 7 wherein said assaying comprises preparing RNA from said cell and optionally using said RNA for quantitative PCR.

17. The method of claim 7 wherein said assaying comprises using an array.

18. The method of claim 7 wherein said cell is present in a ductal lavage or fine needle aspiration sample, which sample is optionally microdissected to isolate one or more cells suspected of being breast cancer cells.

19. A method to determine the grade of breast cancer in a subject comprising assaying a sample of breast cancer cells of said subject for the expression level(s) of one or more genes listed in Table 2.

20. A method to determine therapeutic treatment for a breast cancer patient based upon said patient's expected survival, said method comprising
determining a survival outcome for said patient by assaying a sample of breast cancer cells from said patient for the expression level(s) of one or more one genes listed in Table 2, 3, 4, and/or 6; and
selecting the appropriate treatment for a patient with such a survival outcome.
